(19) United States
(12) Patent Application Publication
(10) Pub. No.: US 2011/0046691 A1
(43) Pub. Date: Feb. 24, 2011

(54) IMPLANTABLE HEART STIMULATOR DETERMINING LEFT VENTRICULAR SYSTOLIC PRESSURE

(75) Inventors: Anders Björling, Solna (SE); Andreas Blomqvist, Spanga (SE); Karin Järverud, Solna (SE)

Correspondence Address: SCHIFF HARDIN, LLP;PATENT DEPARTMENT, 233 S. Wacker Drive-Suite 6600, CHICAGO, IL, 60606-6473, US

(73) Assignee: St. Jude Medical AB

(21) Appl. No.: 12/990,136

(22) PCT Filed: Jun. 18, 2008
(86) PCT No.: PCT/SE2008/000406

§ 371 Date: Oct. 28, 2010

(51) Int. Cl. A61N 1/36

(52) U.S. Cl. 607/17

(57) ABSTRACT

An implantable heart stimulator has an impedance measurement a cardiogenic impedance waveform using an impedance configuration arranged to measure myocardial contractility of the heart. The heart stimulator further has a calculating unit that calculates an estimate value being related to at least two impedance values of the waveform, or of an average waveform of several consecutive waveforms, during a predetermined time period of the waveform, or average waveform, the calculated estimate value being an estimate of the left ventricular (LV) systolic pressure.

### BACKGROUND OF THE INVENTION

1. Field of the Invention

The present invention relates to an implantable heart stimulator of the type wherein an impedance measurement is made in order to measure myocardial contractility of the heart.

As used herein, the term implantable heart stimulator means any device suitable for generating stimulation pulses to be applied to the heart, e.g. a pacemaker, a cardioverter or a defibrillator.

2. Description of the Prior Art

When delivering pacing therapy with a cardiac device, it is often a problem to know when an optimal cardiac situation has been achieved. There is at this point no apparent way to do this sufficiently well in an automated implementation in an implantable medical device.

Known techniques may optimize different aspects of the cardiac function such as stroke volume or aortic velocity time, but it is in most cases in an ideal cardiac case and the optimizations do not take the hearts own metabolism into account.

Impedance measurements may be a basis for optimizing cardiac function when using an implantable heart stimulator. From US 2007/0191901 A1 it is known to measure various impedance related parameters and use these parameters for programming a cardiac resynchronization therapy (CRT). Mechanical myocardial systole and diastole may be identified by evaluating impedance signals over time, and integration of impedance gives an estimate of cardiac function.

It is commonly known to measure impedance of the heart by using multi-polar electrodes. From U.S. Pat. No. 5,501,702 A it is known to make impedance measurements from different electrode combinations. Measurement of impedance present between two or more sensing locations is referred to as rheography. Rheographic techniques allow measurements of physiological parameters without the need for a special sensor; instead multiple electrodes on a standard pacing lead are used. As shown in the referenced patent, an impedance measurement is made by delivering a constant pulse between two source electrodes, and then measuring the voltage differential between two recording electrodes to determine the impedance there between. Switches for choosing lead conductors for coupling to a current source or detection circuit are operated in timed synchronism with the delivery of a sequence of current pulses from the current source. With a tetra- or quadripolar rheographic arrangement it is thus possible to monitor the patient's stroke volume and heart tissue contractility.

From US 2003/0204212 A1 it is also known to calculate first time derivatives of the impedance change, dZ/dt and that there exists a linear relationship between peak dZ/dt and peak cardiac ejection rate, which is a basis for determining cardiac output. Impedance waveforms from several beats may be averaged together and averaged impedance waveform changes may be derived. The AV-interval is then changed to find the maximum or minimum impedance waveform change, and the AV-interval giving optimal cardiac output may then be determined.

One way of determining a cardiac situation is to measure the stroke work. In US 2005/0096706 A1 the intracardiac impedance is measured and stroke volume is estimated using the impedance measurement. The ventricular pressure is further measured, and the pressure and the stroke volume forms a pressure-volume loop (PV loop), which area represents the stroke work.

US 2007/0150017 A1 discloses a device and method for improving cardiac efficiency. The object of the device and method therein is to control therapy applied to the heart by minimizing myocardial oxygen consumption for a given external workload, in order to optimize cardiac efficiency. A cardiac efficiency may be calculated by using a measured stroke volume, pulse pressure, heart rate and an oxygen saturation value. Cardiac output may be defined as the product of heart rate or pulse pressure and stroke volume. The stroke volume may be measured by use of intracardiac measurements, the pulse pressure is typically measured using dedicated pressure sensors.

To achieve an optimal cardiac situation, it is important to make as correct measurements and estimates as possible.

### SUMMARY OF THE INVENTION

Thus, one object of the present invention is to achieve an improved device to determine left systolic pressure of the heart. And an additional object is to achieve an improved estimation of the stroke work of the heart for a patient with an implantable heart stimulator.

This object is achieved in accordance with the present invention by an implantable heart stimulator having an impedance measurement circuit that measures a cardiogenic impedance waveform using an impedance configuration arranged to measure myocardial contractility of the heart. The heart stimulator further has a calculating unit that calculates an estimate value being related to at least two impedance values of the waveform, or of an average waveform of several consecutive waveforms, during a predetermined time period of the waveform, or average waveform, the calculated estimate value being an estimate of the left ventricular (LV) systolic pressure.

According to another embodiment of the present invention the heart stimulator further has a second impedance measurement means that determines at least one cardiac stroke volume parameter indicative of the stroke volume of the heart. Then the calculating unit is further adapted to calculate the stroke work of the heart based on the product of the measured cardiac stroke volume parameter and the estimated LV systolic pressure.

As discussed in the background section a major advantage of using impedance measurements to measure pressure is that no extra hardware has to be arranged, i.e. no pressure sensor has to be arranged at the electrode lead which may result in a more complex circuitry and often thicker leads.

In summary, the present invention is designed to determine the systolic pressure by impedance measurements and to use the determined systolic pressure values, either to calculate the stroke work for e.g. optimizing pace parameters and/or lead position in an implantable (CRT) pacemaker, or to use the systolic pressure on its own for e.g. trending and optimization purposes.

### BRIEF DESCRIPTION OF THE DRAWINGS

FIG. 1 is a schematic block diagram illustrating a first embodiment of the present invention.

FIG. 2 is a schematic block diagram illustrating a second embodiment of the present invention.

FIG. 3 is a time graph illustrating the measured impedance signal.

FIG. 4 is a PV diagram illustrating the stroke work calculated according to the present invention.

FIGS. 5 and 6, respectively, show graphs of measured left ventricular pressure (LVP) (top graph) and impedance values (bottom graph) processed according to the present invention.

### DESCRIPTION OF THE PREFERRED EMBODIMENTS

Accordingly, an object with the present invention is to estimate the systolic pressure in a patient with an implanted cardiac device, such as a pacemaker. A further object is to estimate the stroke work in a patient with an implanted cardiac device, such as a pacemaker

By using one impedance configuration and clever signal processing an estimate of the systolic pressure is acquired. By using two different impedance configurations and clever signal processing a correlate of the stroke work may be acquired.

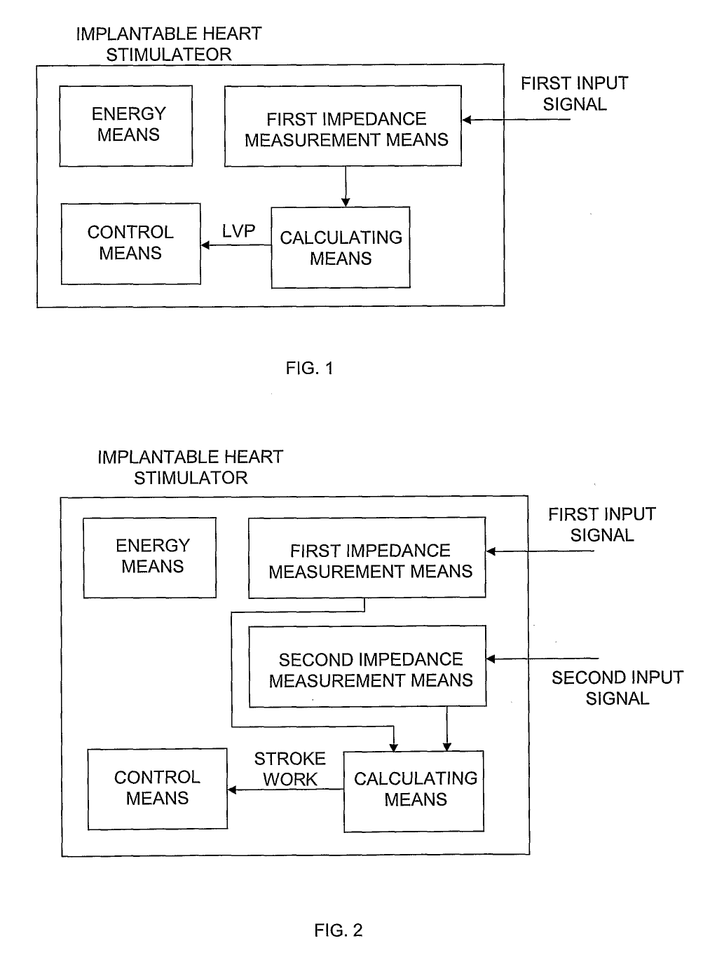

With references to FIG. 1 the present invention is illustrated and relates to an implantable heart stimulator comprising a first impedance measurement means adapted to measure and determine a cardiogenic impedance waveform using an impedance configuration arranged to measure myocardial contractility of the heart. The impedance configuration may be a bipolar left ventricular (LV) configuration or a bipolar right ventricular (RV) configuration using the same electrode leads as being used for LV or RV stimulation. The impedance measurement may also be performed by using an indifferent electrode at the pacemaker can in combination with intracardial electrodes, or any other configuration that may measure myocardial contractility of the heart.

The heart stimulator further has a calculating unit that calculates an estimate value being related to at least two impedance values of the waveform, or of an average waveform of several consecutive waveforms, during a predetermined time period of the waveform, or average waveform. The calculated estimate value is an estimate of the left ventricular (LV) systolic pressure.

In addition the heart stimulator has control means and energy means. The control unit includes, inter alia, necessary circuitry (not shown) that is needed to initiate and generate stimulation pulses. The circuitry may include timing means and storage means. The control means also includes telemetry means (not shown) used for telemetry communication with an external programming means (not shown). The stimulation pulses are applied to the heart tissue via one or many electrode leads (not shown) positioned in one or many chambers of the heart, which may be arranged both in the left and right side of the heart, and in the coronary veins of the heart.

The duration of the predetermined time period, the window length, w, is such that it spans the early systolic phase of the heart cycle. Conventionally the early systolic phase is defined as the phase of the isovolumic contraction (IVC), and starts with the mitral valve closure (MVC) and ends with the aortic valve opening (AVO). According to one embodiment, the predetermined time period is the early systolic portion of the impedance waveform, e.g. initiated by the R-wave.

In another embodiment the predetermined time period is initiated by the R-wave and is terminated by the aortic valve opening. The length of the time period may also be influenced by the age or state of health etc. of the person in question.

The time period length can either be set to a fixed, predetermined value, in the range of 50-400 ms, or it can be flexible.

If it is flexible, the value is set so that T_{R}+w occurs either at
1) the time of the maximum value of the first derivative of an Z signal in the 400 ms following the R wave,
   or at
2) the time of the maximum value of the second derivative of an Z signal in the 400 ms following the R wave.
   T_{R} is the starting point of the time window.

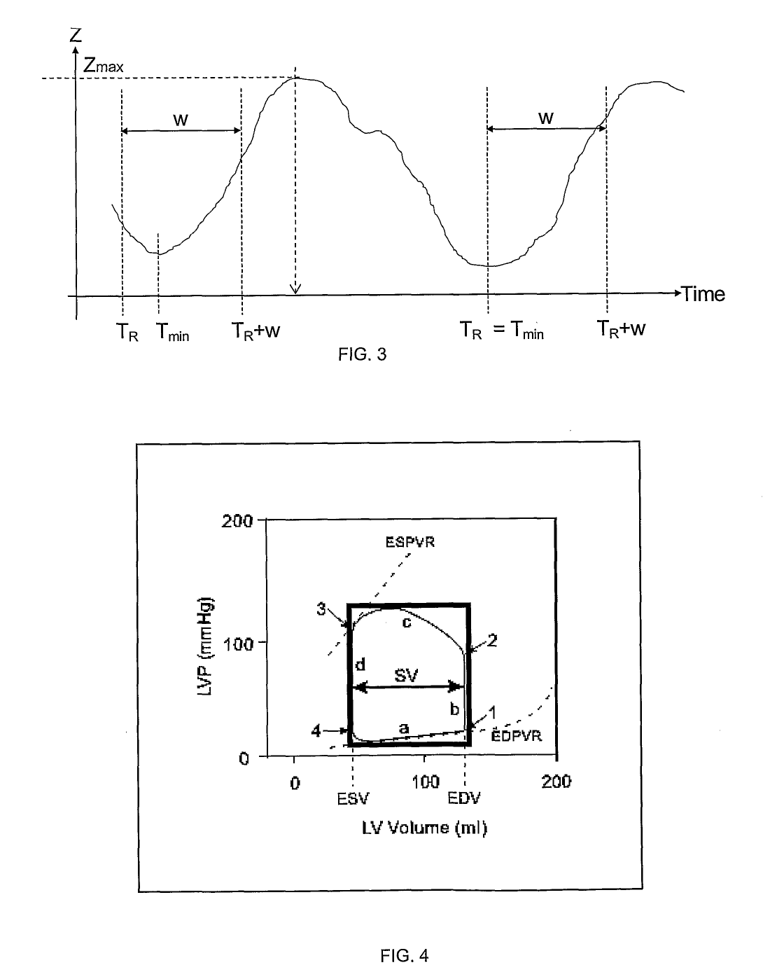

In still another embodiment the predetermined time period is identified during a time window initiated by the R-wave and terminated when the impedance value Zₘₐₓ is maximal. The time when Zₘₐₓ occurs may be determined by applying a conventional pattern recognition or morphology recognition technique of the impedance signal to identify the maximum value and the corresponding point of time. This is schematically illustrated in FIG. 3.

The inventors have seen in simulation models as well as in preclinical studies that the very early phase of the dφ)/dt (with φ representing the aortic blood flow) correlates very well with dP/dt. It has also been shown that the rate of change in cardiogenic impedance (dZ/dt) following the QRS correlates very well with dφ/dt. Hence it is assumed that the dZ/dt is a good estimate of dP/dt, in the systolic part of the heart cycle in the left ventricle (LV). The integral of dZ/dt would then yield the systolic pressure.

The following mathematical formulae describe the relations:

$$\frac{d z}{d t} {\vert}_{t = {T}_{R} … {T}_{R} + w} ∝ \frac{d P}{d t} {\vert}_{t = {T}_{R} … {T}_{R} + w} ⇒ \\ {\int }_{{T}_{R}}^{{T}_{R} + w} \frac{d z}{d t} d t = ( z ( {T}_{R} + w ) - z ( {T}_{R} ) ) ∝ … \\ \begin{matrix}… ( P ( {T}_{R} + w ) - P ( {T}_{R} ) ) \approx ( P ( {T}_{R} + w ) - 0 ) \\ = P ( {T}_{R} + w ) \\ ≡ {P}_{systolic}\end{matrix} \\ ∵ {P}_{systolic} ∝ z ( {T}_{R} + w ) - z ( {T}_{R} )$$

where P is the pressure, w is the window length and T_{R} is the time of the R wave. FIG. 3 schematically shows an impedance waveform during almost 2 complete heart cycles.

In other words, an estimation of the left ventricular pressure may be calculated, according to the formula above, as the impedance value at the time T_{R}+w minus the impedance value at the time T_{R}.

Usually, the lowest pressure does not occur at the exact time of the R wave. Thus, the minimum value of the impedance does not align in time with the time of the R wave. In the formulae above it is partly assumed that this is the case, thus the correlate of the systolic pressure can be estimated in two slightly different ways:

Either P_{systolic}∝z(T_{R}+w)−z(T_{R}) or P_{systolic}∝z(T_{R}+w)−z(Tₘᵢₙ)

The procedure, according to one embodiment, for estimating the LV pressure is summarized in the following steps:
1. Measure the cardiogenic impedance in an impedance configuration that is influenced by the myocardial contractility of the LV, e.g. LV bipolar or RV bipolar.
2. Calculate the average of a few heart cycles, e.g. 10, to produce an average impedance waveform. One heart cycle is defined as going from one R wave to the subsequent R wave as detected by the IEGM acquired by the device. It is important that the averaging spans over a complete breathing cycle, as this influence the impedance.
3. Calculate the entity z(T_{R}+w)−z(T_{R}) or Z(T_{R}+w)Z(Tₘᵢₙ) and store this as the systolic pressure estimate.

Thus, according to one embodiment the estimate value being the difference between the two impedance values within the predetermined time period.

As is illustrated above in the first alternative of (3) the used impedance values being the respective impedance values at the beginning and at the end of the predetermined time period.

As illustrated above in the second alternative of (3) the used impedance values being the minimum impedance value during the predetermined time period and the impedance value at the end of the predetermined time period, respectively.

According to a further alternative the estimated LV systolic pressure is calculated by integrating the rate of change (dZ/dt) of the calculated waveform during the predetermined time period.

In order to increase the calculation accuracy waveforms from several heart cycles are used. Two different calculation alternatives may then be used, either an average waveform is calculated from several heart cycles and an estimate of the systolic pressure is calculated from the average waveform, or an estimate of the systolic pressure is calculated for each separate heart cycle and an average estimate of the systolic pressure is then calculated for these separate estimates.

The average waveform is calculated of recorded cardiogenic impedance waveforms during at least one complete breathing cycle.

The calculated left ventricular (LV) systolic pressure is stored in the storage means and long-term trends may be determined and analysed, either by the control means, or the pressure values may be transferred via the telemetry means to the external programming device for further analysis.

Now with references to FIG. 2 another embodiment of the present invention is illustrated where the heart stimulator, in addition to the features illustrated in FIG. 1 further comprises a second impedance measurement means adapted to determine at least one cardiac stroke volume parameter indicative of the stroke volume of the heart. The calculating unit further is adapted to calculate the stroke work of the heart based upon the product of the measured cardiac stroke volume parameter and the estimated LV systolic pressure. Stroke work is defined as the work done by the ventricle to eject a volume of blood (i.e. stroke volume) into the aorta. The cardiac work may also be calculated, which is the product of stroke work and heart rate.

Thus, in order to calculate the stroke work the stroke volume must first be determined.

In an ongoing human study, as well as in numerous pre-clinical studies, strong support have been identified that the peak to peak value of the cardiogenic impedance recorded over the left ventricle correlates well with stroke volume (or cardiac output). By averaging a number of heart cycles—this to remove noise and respiration—it is possible to estimate the stroke volume.

The algorithm then is composed of three simple steps:
1. Measure the impedance using a vector that spans across the left ventricle, e.g. RV-LV quadropolar. It is also possible to measure the impedance in a tripolar fashion involving RV and LV leads and/or the can.
2. Calculate the average of a few heart cycles, e.g. 10, to produce an average impedance waveform. One heart cycle is defined as going from one R wave to the subsequent R wave as detected by the IEGM acquired by the stimulator. It is important that the averaging spans over a complete breathing cycle, as this influence the impedance
3. Find the peak to peak value of this averaged impedance waveform. This value correlates with stroke volume

The above procedure for identifying the stroke volume is to be regarded only as one example of many available ways to identify the stroke volume by using impedance measurements, see e.g. the above-mentioned US 2005/0096706 A1.

In the calculation of the stroke work, two different impedance configurations are used: one used for assessing the volume of the heart and one for assessing the pressure.

In the estimation of the stroke work, the estimation of the stroke volume and the estimation of the systolic pressure are multiplied.

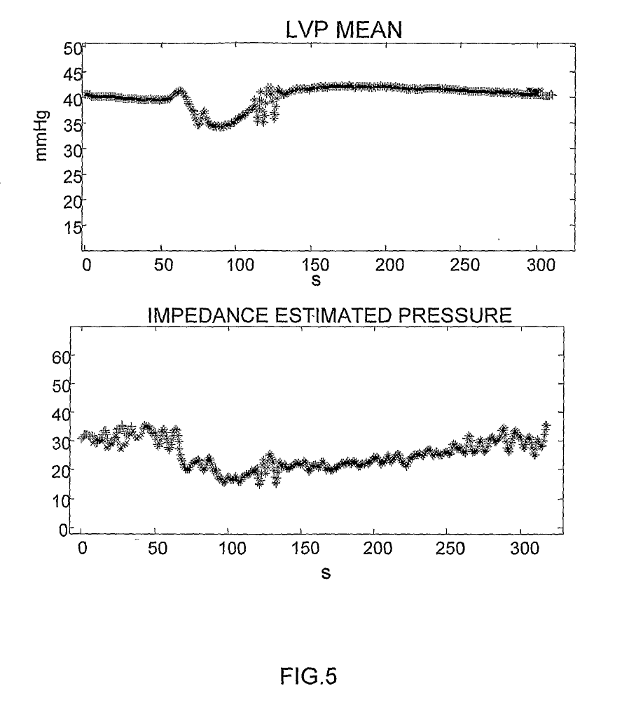

FIG. 4 shows a so-called PV loop. In FIG. 4 EDV denotes end diastolic volume, ESV denotes end systolic volume, ESPVR denotes end systolic pressure-volume relationship and EDPVR denotes end diastolic pressure-volume relationship. Further, LVP denotes left ventricular pressure in mmHG and LV Volume denotes the volume of the left ventricle in ml.

The true stroke work equals the area that is enclosed by curves a, b, c and d. The curves represent the four basic phases of a heart cycle: curve a equals the ventricular filling phase, b equals the isovolumetric contraction phase, c the ejection phase and d the isovolumetric relaxation phase. The numbers 1-4 in the figure indicates different transition points run through during one heart cycle. The width of the PV-loop represents the difference between EDV (end diastolic volume) and ESV (end systolic volume), which by definition is the stroke volume (SV). The calculated estimate of the stroke work correlates to the area of the rectangular box. During short time periods, the sizes of the rectangular and true stroke work areas correlate very well, i.e. during a short optimization situation it is believed that the correlation between the true stroke work and the pressure-volume-product to be high enough to give a good estimate of the stroke work.

In one embodiment the calculated stroke work is used to optimize settings of the heart stimulator, e.g. such that the stroke work is maximized (the higher the stroke work correlate or the higher the systolic pressure, the better). The optimization may be performed by continuously, or at follow-up, change the AV-delay, the VV delay, the pacing configuration, the base rate etc.

In another embodiment the calculated stroke work is used to optimize lead position.

An optimal lead placement is evaluated by running through a sequence of different combinations of VV delays, AV delays, base rates, pacing vectors and other device parameters with the leads at different positions. At implant, the physician would then place the leads in different positions and the implant, or programming device connected to the leads, would then, e.g. automatically, determine the optimal lead position based upon the position yielding the highest stroke work value.

It is also possible to arrange a left ventricular lead with several electrodes that can be selected individually by electronic means. This makes it possible to optimize electrode position post implant.

In still another embodiment the calculated stroke work is stored and trended. The stroke work is stored in the storage means of the control means, where it also is further analyzed. As an alternative, the stroke work values are transmitted via telemetry to an external programming device for further analysis. The analysis may be tailored to specify specific situations of particular interest, e.g. the trend analysis may be performed during a predetermined time period at a given level of activity for the patient. The trend analysis of the systolic pressure correlate may be reported to a physician, the trend analysis is interesting in itself and for e.g. drug titration.

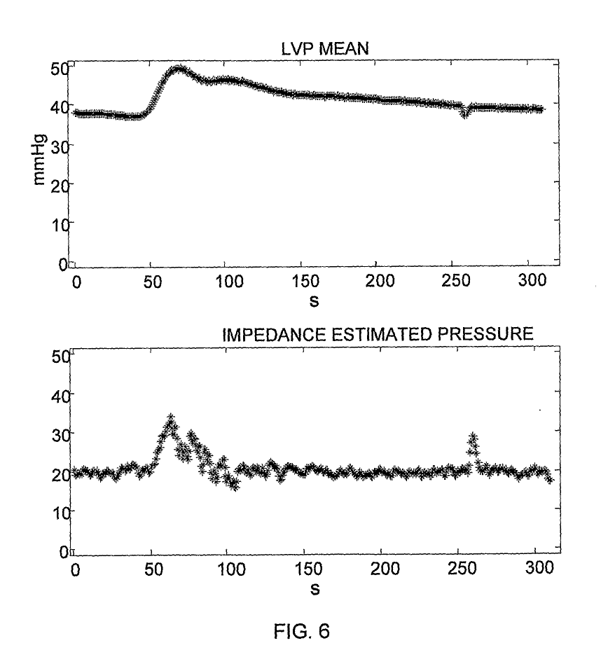

FIGS. 5 and 6, respectively, show graphs of measured left ventricular pressure (LVP) (top graph) and impedance values (bottom graph) processed according to the present invention. LVP was recorded in an acute setting in porcine subjects. Data included here was acquired during infusion of dobutamine. The LVP was recorded using a commercial pressure sensor (Millar catheter) and the impedance was processed according to the present invention. The impedance configuration for performing the impedance measurements is the RV-bipolar.

The impedance parameter used to estimate pressure shows a good correlation to the real pressure values and time synchronized response to provocation. It is understood that the impedance values have to be calibrated to be comparable to the real pressure values by value.

As an illustration of how the impedance parameter is calculated one example of a possible calculation code is shown in the following:

```
function [values,min_val,max_val,mid_val]=A08E2007(Z,pos_vec)
Z = gausssmooth(Z,11);
for jj=1:length(pos_vec)−1
  excerpt=Z(pos_vec(jj):pos_vec(jj+1)−1);
  dZ = gradient(excerpt);
  dZ=gausssmooth(dZ,5);
  [Y,I]=max(dZ);
  min_val(jj) = min(excerpt(1:l)); max_val(jj) = excerpt(l);
  value = max_val(jj)−min_val(jj); values(jj) = value;
end
```

Here the impedance curve Z is input together with the desired predetermined time interval, pos_vec. The values, min_val and max_val are derived, and a value being the difference between the max_val and the min_val is calculated which is the impedance estimated pressure. The total estimated impedance parameters are gathered in a vector, values (jj), after iteration, and shown in the lowermost plots of FIGS. 5 and 6.

Although modifications and changes may be suggested by those skilled in the art, it is the intention of the inventors to embody within the patent warranted heron all changes and modifications as reasonably and properly come within the scope of their contribution to the art.

We claim as our invention:

1.-16. (canceled)

17. An implantable heart stimulator comprising:
an impedance measurement circuit that measures and determines a cardiogenic impedance waveform with an impedance configuration that measures myocardial contractility of the heart; and
a calculating unit supplied with said cardiogenic impedance waveform, said calculating unit being configured to calculate an estimate of left ventricular systolic pressure of said heart from two impedance values of said cardiogenic impedance waveform, selected from the group consisting of at least two impedance values of a single waveform, and at least two impedance values of an average waveform of a plurality of consecutive waveforms, during a predetermined time period of said cardiogenic impedance waveform, and to emit the calculated estimate value as an output from said calculating unit.

18. An implantable heart stimulator as claimed in claim 17 wherein said calculating unit is configured to calculate said estimate value as a difference between said at least two impedance values.

19. An implantable heart stimulator as claimed in claim 17 wherein said calculating unit selects said at least two impedance values respectively at a beginning and an end of said predetermined time period.

20. An implantable heart stimulator as claimed in claim 17 wherein said calculating unit selects said at least two impedance values as the minimum impedance value that occurs during said predetermined time period, and an impedance value at an end of said predetermined time period, respectively.

21. An implantable heart stimulator as claimed in claim 17 wherein said calculating unit is configured to calculate an estimated left ventricular systolic pressure by integrating a rate of change of said waveform during said time period.

22. An implantable heart stimulator as claimed in claim 17 wherein said calculating unit is configured to select said predetermined time period during an early systolic portion of said waveform.

23. An implantable heart stimulator as claimed in claim 17 wherein said calculating unit is configured to determine said predetermined time period as a time period initiated by an R-wave and terminated by a subsequent aortic valve opening.

24. An implantable heart stimulator as claimed in claim 17 wherein said calculating unit is configured to determine said predetermined time period as a time period initiated by an R-wave and lasting for a time duration thereafter in a range between 50 and 200 ms.

25. An implantable heart stimulator as claimed in claim 17 wherein said calculating unit is configured to determine said predetermined time period as a time window initiated by an R-wave and terminating when an impedance value Zₘₐₓ is maximum, a time when Zₘₐₓ occurs being determined by morphology analysis of said cardiogenic impedance signal to identify said maximum value and a corresponding point in time.

26. An implantable heart stimulator as claimed in claim 17 wherein said calculator employs said average waveform as said cardiogenic impedance waveform, and calculates said average waveform from detected cardiogenic impedance waveforms during at least one complete respiration cycle.

27. An implantable heart stimulator as claimed in claim 17 comprising a memory in which successively calculated left ventricular systolic pressures emitted from said calculating unit are stored, and a processor having access to said memory configured to calculate a trend represented by the respective left ventricular systolic pressures stored in said memory.

28. An implantable heart stimulator as claimed in claim 17 wherein said impedance measurement circuit is a first impedance measurement circuit, and comprising a second impedance measurement circuit that determines at least one cardiac stroke volume parameter indicative of the stroke volume of the heart, and wherein said calculating unit is configured to calculate stroke work of the heart based on a product of said measured cardiac stroke volume parameter and the estimated left ventricular systolic pressure.

29. An implantable heart stimulator as claimed in claim 28 wherein said heart stimulator comprises a therapy unit configured to administer stimulation therapy to the heart according to settings, and comprising a computerized control unit supplied with the calculated stroke work, said computerized control unit being configured to optimize said settings dependent on the calculated stroke work to maximize the stroke work of the heart.

30. An implantable heart stimulator as claimed in claim 29 comprising a stimulation therapy administration circuit comprising at least one therapy-administering electrode lead, exhibiting a lead position, and comprising a computerized control unit that controls said therapy administration circuit dependent on the calculated stroke work to optimize said lead position.

31. An implantable heart stimulator as claimed in claim 29 comprising a memory in which the calculated stroke work, calculated at respectively different times, is stored, and a processor having access to said memory configured to determine a trend represented by the calculated stroke work at said different times stored in said memory.

32. An implantable heart stimulator as claimed in claim 31 wherein said processor is configured to trend the calculate stroke work over time at a predetermined level of activity of a patient.
